(19) United States
(12) Patent Application Publication
(10) Pub. No.: US 2004/0171128 A1
(43) Pub. Date: Sep. 2, 2004

(54) Halogenated emulsans

(76) Inventor: Manssur Yalpani, Rancho Sante Fe, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 32606-6669, US

(21) Appl. No.: 10/790,884

(22) Filed: Mar. 1, 2004

Related U.S. Application Data
(60) Provisional application No. 60/450,653, filed on Feb. 28, 2003.

(51) Int. Cl. C12P 7/64; C12N 1/21

(52) U.S. Cl. 435/134; 435/252.3

(57) ABSTRACT

Halogenated emulsans are prepared by a fermentation process where a microorganism capable of making emulsans is grown (cultured) in an effective emulsan producing culture medium with a suitable carbon source and halogenated compounds that are assimilated by the microorganism and incorporated into the emulsan molecule made by the microorganism. The emulsans are secreted into the culture medium and isolated therefrom. A preferred microorganism is a bacterium from the genus Actinobacter and preferred halogenated compounds include fluorinated compounds such as fluorinated fatty acids and fluorinated fatty acid esters.

### CROSS-REFERENCED TO A RELATED APPLICATION

This application claims the benefit of U.S. provisional application Serial No. 60/450,653, filed Feb. 28, 2003.

### FIELD OF THE INVENTION

The present invention relates to halogenated biopolymer derivatives useful as biosurfactants, antimicrobial agents, imaging probes, diagnostic agents and contrast agents and to methods of making and using the halogenated biopolymers.

### BACKGROUND OF THE INVENTION

Emulsans are amphipathic polysaccharides that stabilize oil-in-water emulsions. They are produced by a wide diversity of microorganisms. The bacterium Acinetobacter calcoaceticus RAG1 produces emulsan, an amphilphilic, anionic exopolysaccharide bearing ester- and amide-linked fatty acid groups. Emulsan's main chain comprises three aminosugars, D-galactosamine, D-galactosaminouronic acid, and a dideoxy-diamino hexose in a 1:1:1 ratio. The polymer has O-acyl and N-acyl bound side chain fatty acids ranging in chain length from C₁₀ to C₁₈ (see FIG. 1), respectively. These fatty acid substituents constitute up to 23% (w/w) of the polymer. The emulsan amino groups are either acetylated or covalently linked by an amide bond to 3-hydroxybutyric acid. The fatty acid composition of emulsan and thus its emulsifying properties can be modulated by feeding strategies (Zhang J., Lee S.-H., Gross R. A., Kaplan D. L., J. Chem. Techn. Biotechn., 74, 759-765, 1999.) and genetic modifications. In general, emulsans can be generated in high yields on a wide range of carbon sources, including ethanol and fatty acids (Gutnick D. L. et al. (eds.), Exopolysaccharide Bioemulsifiers, Marcel Dekker, New York, Vol. 25, pp. 211-246, 1987; Gutnick, D. L. et al. (eds.), Exopolysaccharide Bioemulsifiers, Marcel Dekker, Inc.: New York, Vol. 25, pg. 232, 1987; Belsky, I., Gutnick D. L., Rosenberg E., FEBS Lett., 101, 175-178, 1979.). The combination of hydrophilic anionic carbohydrate backbone and the hydrophobic branches leads to Emulsan's amphipathic behavior and ability to form stable oil-in-water emulsions. The polymer is biodegradable, and its solution properties are modulated by the nature of the fatty acid pendant groups. Emulsan causes little reduction in surface tension between an oil and water interface (10 dynes/cm), but binds tightly to oil surfaces, protecting oil droplets from coalescence. Emulsan emulsions are stable for months and can be broken down enzymatically or at elevated temperatures (e.g., >50° C.). Emulsans have potential applications in agriculture, cosmetics, pharmaceuticals, detergents, personal care products, food processing, textile manufacturing, laundry supplies, metal treatment and processing, pulp and paper processing and paint industries.

Emulsans offer several advantages over conventional emulsifiers and surfactants presently used in industry. Typical commercial synthetic surfactants are usually toxic to the environment and non-biodegradable. They may bioaccumulate and their production, processes and by-products can be environmentally hazardous. Biosurfactants, like emulsans, have advantages over their chemical counterparts in biodegradability and effectiveness at extreme temperature or pH and in having lower toxicity. Biosurfactants are mainly used in studies on enhanced oil recovery and hydrocarbon bioremediation. The solubilization and emulsification of toxic chemicals by biosurfactants have also been reported.

### BRIEF SUMMARY OF THE INVENTION

The present invention relates to halogenated biopolymer emulsan derivatives of Formulas I useful as biomaterials. Additionally, the present invention relates to methods of making and using the present compounds of Formula I. Formula I is as follows:

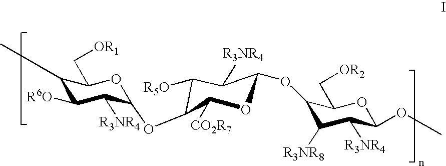

Where

R₁═H, acyl, X; R₂═H, acyl, X; R₃═H, COCH₃, X; R₄═H, X COX, CH₂X, CH₂NHX; R₅═H, X; R₆═H, X; R₇═H, X, COCH₃, COX

X=haloalkyl, haloaryl, haloacyl, perhaloalkyl, perhaloaryl, perhaloacyl, perhalopolymer fluoroalkyl, fluoroaryl, fluoroacyl, perfluoroalkyl, perfluoroaryl, perfluoroacyl, perfluoropolymer, F, Cl, Br, I, CF₃, CCl₃ COCₓF_{y}, CH₂Br, CH₂I, CₓF_{y}H_{z}, COCH₂(CₓH_{3-y}Br_{y}), COCH₂(CₓH_{3-y}Cl_{y}), COCH₂(CₓH_{3-y}I_{y}), CₓF_{y}H_{z}, ([CH₂]ₘO)ₓ(CH₂CF₂O)_{y}(CF₂CF₂O)_{z}(CF₂)₂CF₂CH₂O(CH₂)ₚOH, CH₂C(OH)CₓF_{y}H_{z}, CₓF_{y}H_{z}Oₚ, COCₓF_{y}H_{z}, OCH₂CₓF_{z}[CₓF_{z}O]ₘF, CH₂C(CH₃)CO₂CₓH_{z}(CF₂)ₘCF₃, CH₂(CF₂O)ₓ(CF₂CF₂O)_{y}(CF₂O)_{z}CF₂CH₂OH, COCF(CF₃)-[CF(CF₃)CF₂O]ₘF, NHCₓF_{y}H_{z}Oₚ, CH₂CF₂O[CF₂CF₂O]ₘ(CF₂OCF₂CH₂OH, COCₓH_{z}(CF₂)ₘCF₃, COCF₂O[CF₂CF₂O]ₙCF₂OCF₂CO₂H, ([CH₂]ₘO)ₓ(CH₂CF₂₀)_{y}(CF₂CF₂O)_{z}CF₂CH₂O(CH₂)ₚOH, N[CₓF_{y}H_{z}]ₚ, CₓH_{z}CO₂CₓH_{z}(CF₂)ₘCF₃, COCₓF_{y}[CₚF_{z}O]ₘF, a luminescent residue, a fluorescent residue, a halogenated (such as a fluorinated) luminescent residue or a halogenated (such as a fluorinated) fluorescent residue and m, x, p, y and z are integers from 1 to 150, and where m is preferably 10-100, and more preferably 10-50, and where x, p, y, z are preferably 10-75, more preferably 10-50, and most preferably 10-20. Acyl and alkyl residues in the above formulas comprise lipophilic moieties, including saturated and unsaturated aliphatic residues with Cₖ chains, where k is 2 to 100, preferably 250, and more preferably 2-20, and aryl residues comprise aromatic moieties, including benzyl, biphenyl, phenyl polycyclic aromatics, and heteroatom-containing aromatics.

The present invention is directed to novel compositions comprising the halogenated emulsan biopolymers of Formula I for use as new biomaterials, such as drug delivery agents, antimicrobial agents, emulsifiers, emulsion stabilizers, biosurfactants, imaging probes, diagnostic tools and contrast agents. Fluorinated emulsan biopolymers are preferred.

The present invention is also directed to a fermentation process to make the present halogenated emulsans. A microorganism capable of making emulsans is grown (cultured) in an effective emulsan producing culture medium with a suitable carbon source and halogenated compounds that are assimilated by the microorganism and incorporated into the emulsan molecule made by the microorganism. The emulsans are secreted into the culture medium and isolated therefrom. A preferred microorganism is a bacterium from the genus Actinobacter and preferred halogenated compounds include fluorinated compounds such as fluorinated fatty acids and fluorinated fatty acid esters.

Of particular interest in practicing the present invention, Actinobacter calcoaceticus RAG1 (hereinafter RAG1) is fermented employing ethanol as a carbon source as well as fluorinated fatty acids or fluorinated fatty acid esters whereby the fluorinated fatty acids and/or esters are assimilated by the RAG1 and incorporated into the emulsan molecule formed by the RAG1. Preferred fluorinated compounds include 2-poly (perfluoro propanoxy)-methyl perfluoro propanoate (hereinafter “16F”) or 2-[-(carboxy-difluoromethoxy)-poly(perfluoro methoxy)]-poly(perfluoro ethanol)-difluoro acetic acid (hereinafter “18F”). The fluorinated emulsans that contain 16F and 18F attached to the polysaccharide backbone possess emulsification activity.

The present invention can also be viewed as an improvement to the process of making emulsans by culturing an emulsan producing microorganism in a culture medium containing a carbon source under conditions sufficient to produce emulsans wherein the improvement comprises employing as the carbon source a lower alcohol and one or more halogenated compounds whereby the halogenated compounds are assimilated by the microorganism and incorporated into the emulsan molecule resulting in halogenated emulsans. Preferably the improved process involves the preparation of fluorinated emulsans where the halogenated compounds comprise fluorinated compounds. A preferred lower alcohol is ethanol and preferred fluorinated compounds include 16F and 18F.

### BRIEF DESCRIPTION OF THE FIGURES

FIG. 1 shows an Emulsan structure

FIG. 2 ¹⁹F-NMR of (a) native emulsan, (b) 2-poly(perfluoropropanoxy)-methylperfluoropropanoate, (c) incorporation of the compound in ‘b’ into emulsan, (d) monomer polytetrafluoroethyleneoxide-co-difluoromethyleneoxide-α,ω-bis(methylcarboxylate), and (e) incorporation of compound ‘d’ into emulsan.

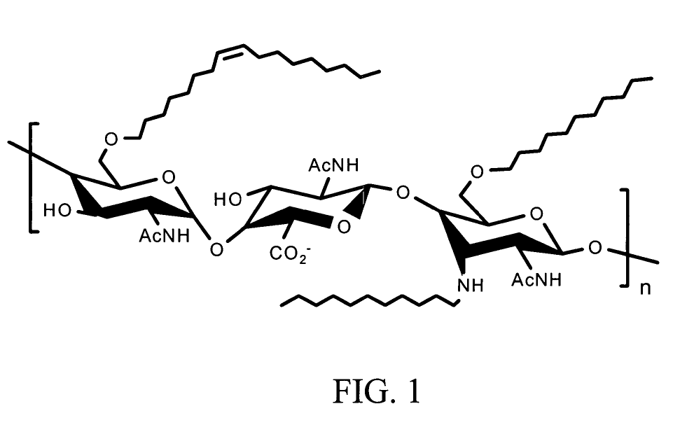

### DETAILED DESCRIPTION OF THE INVENTION

Preparation of new Halogenated Emulsans

Compositions of this invention are obtained by fermentation processes that employ suitable halogenated substrates, such as those described herein.

These approaches permit the preparation of halogenated biopolymers with a broad range of halogen substituent types and incorporation levels (5-40% as illustrated in the following Examples) that can be tailored to either industrial, diagnostic or therapeutic uses. The optimum halogen content will be determined in each case by the use requirements for sensitivity on one hand and the extent to which the maximum halogen substitution does not interfere with the probe's biological or physicochemical properties, e.g., its solubility or receptor binding ability. An important parameter in these considerations will be the nature and level of halogen substitution. Generally preferable halogen levels are 10-40%, and more preferable 20-40%.

A preferred embodiment of the present invention permits the preparation of fluorinated biopolymers with a broad range of fluorine substituent types and incorporation levels (5-40% as illustrated in the following Examples) that can be tailored to either industrial, diagnostic or therapeutic uses. The optimum fluorine content will be determined in each case by the use requirements for sensitivity on one hand and the extent to which the maximum fluorine substitution does not interfere with the probe's biological or physicochemical properties, e.g., its solubility or receptor binding ability. An important parameter in these considerations will be the nature and level of fluorine substitution. Generally preferable F levels are 10-40%, and more preferable 20-40%.

Suitable fluorinated starting materials for making the novel compositions of the present invention include, but are not limited to fluoroalkylcarboxylic acids, fluoroalkylaldehydes, anhydrides, esters, ketones, acid chlorides of fluoroalkylcarboxylic acids, such as monofluoroacetic acid, difluoroacetic acid, trifluoroacetic acid, pentafluoropropionic acid, heptafluorobutyric acid, heptafluorobutyric anhydride, hepta-fluorobutyrylchloride, nonafluoropentanoic acid, tridecafluoroheptanoic acid, pentadecafluorooctanoic acid, heptadecafluorononanoic acid, nonadecafluorodecanoic acid, perfluorododecanoic acid, perfluorotetradecanoic acid; fluoroalkanols, such as 2,2,3,3,4,4,4-heptafluoro-1-butanol, 2,2,3,3, 4,4,5,5,6,6,7,7,8,8,9,9,10,10,11,11,11-heneicosafluoro-1-undecanol, 2,2,3,3,4,4,5,5,6,6,7,7, 8,8,9,9-heptadecafluoro-1-nonanol, 2,2,3,3,4,4,5,5,6,6,7,7,8,8,8-penta-decafluoro-1-octanol, 2,2,3,3,4,4,5,5,6,6, 7,7,8,8,9,9,10,10,10-nonadeca-fluoro-1-decanol, Krytox and Zonyl derivatives, fluoroarylesters, fluoroalkylamines, fluoroarylamines, fluorinated polymers containing reactive terminal groups, fluoroalkyl halides, such as perfluoroethyl iodide, perfluoropropyl iodide, perfluorohexyl bromide, perfluoroheptyl bromide, perfluorooctyl bromide, perfluorodecyl iodide, perfluorooctyl iodide, 1,1,1,2,2, 3,3,4,4,5,5,6,6,7,7,8,8-heptadecafluoro-10-iododecane, 1,1,1,2,2,3,3,4,4,5,5,6,6,7,7,8,8-heptadeca-fluoro-10-iododecane, polytetrafluoroethyleneoxide-co-difluoromethyleneoxide-α,ω-bis(methylcarboxylate), dihydroxypropanoxymethyl derivatives of perfluoropolyoxyalkane, hydroxypolyethylenoxy derivatives of perfluoropolyoxyalkane and the like. Additionally, analogous non-fluorine halogenated starting materials are employed to make additional halogenated emulsans.

The compounds used in the method of the invention can be prepared readily according in the following detailed examples using readily available starting materials, reagents and conventional synthetic and fermentation procedures. The following examples illustrate the practice of the present invention but should not be construed as limiting its scope.

Preparation of Fluoroemulsans

Strain and culture conditions

A. calcocaceticus RAG-1 (ATCC 31012) was obtained from American Type Culture Collection. Growth of A. calcocaceticus RAG-1 was on defined mineral medium (containing per liter: K₂HPO₄.3H₂O, 22.2 g; H₂PO₄, 7.26 g; MgSO₄.7H₂O, 0.5 g; (NH4)₂SO4, 4 g; EtOH, 1%, and supplemented in separate flasks with 1% (w/v) of each of the fluorinated monomers of study (Table 1). Cultures were grown in 500 mL baffled flasks containing 100 mL media and incubated at 30° C. in an orbital shaker (250 rpm) for 6 days. Cell growth was determined by protein analysis of the cultures. A 200 μl aliquot of medium was withdrawn and centrifuged at 10,000 g for 3 minutes. The pellet was resuspended in 600 μl of 0.2N NaOH, heated for 10 minutes at 100° C., and centrifuged at 10,000 g for 3 minutes. Total protein was determined by the Lowry method using bovine serum albumin as a calibration standard.

Isolation and the Purification of Emulsan

Cultures were harvested by centrifugation for 30 min at 9,500 rpm. The emulsan polymer was precipitated from the supernatant by the addition of ammonium sulfate to approximately 40% saturation while the solution was maintained at 4° C. The precipitated product was isolated by centrifugation, desalted by dialysis (Spectrum, MW cut-off 6,000-8,000 Da) and lyophilized. Residual impurities were removed by Soxhlet extraction with ether. Associated proteins were removed from the polymer by hot phenol extraction (Zuckerberg, A., Diver, A., Rosenberg, E., Gutnick D. L., Appl. Environ. Microbiol., 37,414-420,1979).

Fatty Acids Analysis

Five milligrams of the purified and deproteinated emulsan was added to 2 mL of a 0.45 N KOH solution in methanol/water (90:10, v/v). The mixture was sealed in a Teflon-capped vial and kept at 100° C. under argon for 6 h. After cooling to room temperature, the reaction mixture was diluted with 2 mL water, acidified with 37% HCl to pH 2, and extracted with ether (4×3 mL). The ether extract was dried over MgSO₄, solids were removed by filtration and volatiles were removed by an argon stream. Fatty acid methyl esters were prepared by treating the fatty acid mixture dissolved in dry (KOH-treated) distilled ether (30 mL) with diazomethane. The diazomethane solution was added slowly at room temperature until N₂ evolution ceased and the yellow color persisted. After an additional 15 min at room temperature the ether was removed by a stream of N₂ (Belsky et al., 1979). The product obtained was dissolved in a mixture of 2 mL of chloroform containing 200 μL of 1.5 mg/mL tetradecane as an internal standard, filtered through a 45 μm syringe filter, dried using an argon stream and dissolved in 50 μL of CHCl₃. The fatty acid composition was determined by Gas Chromatography (Hewlett Packard 5980) coupled to a mass selective detector (HP 5988 series). GC was calibrated by determining relative response factors using fatty acid methyl ester standards. The column used was a SE54 capillary column (5% diphenyl, 95% dimethyl polysiloxane, 30 m, i.d., 0.32 mm). The conditions were: 1 min isotherm at 140° C. followed by 5° C./min ramp to 290° C., and then 5 min isothermal at 290° C. A splitless injector (290° C.) and GC interface at 240° C. were used for the analysis. Fluoro fatty acids methyl esters were identified on the basis of their retention time and Electron Impact (EI) and Chemical Ionization (CI) mass spectra.

¹⁹F-NMR: ¹⁹F-NMR spectra were obtained on a Bruker Aspect 2000 DPX-300 spectrometer, operating at 282.58 MHz. Samples and standards were dissolved in D₂O and CDCl₃, respectively.

Emulsification assay:

Emulsification activity was determined by modification of a published procedure (Rosenberg E., Perry A., Gibson D. T., Gutnick D. L., Appl. Environ. Microbiol., 37, 409-413, 1979; Zhang et al., 1999). Mixtures containing 2 mg of the emulsan analog, 0.2 mL of a substrate (hexadecane, tetradecane or dodecane), and 15 mL of 20 mM Tris/10 mM MgSO₄ (pH 7.2) were introduced into 100 mL baffled flasks and incubated at 30° C. with shaking at 150 rpm for 1 h. Turbidity of the assay mixtures was assayed with a Klett-Summerson colorimeter (green filter) after allowing the mixture to settle for 10 min. Emulsification activity was expressed as turbidity in Klett units (Zhang et al. 1999).

### EXAMPLES 1-11

A. calcocaceticus RAG-1 grew on the majority of the fluorinated substrates screened (Table 1). This was growth in the presence of 1% ethanol, as initial studies revealed no growth on any of the fluorinated substrates in the absence of ethanol. However, retention of the fluorine in the emulsan polymers was only found on two of the substrates, 2-poly(perfluoropropanoxy)-methylperfluoropropanoate, and 2-[(carboxydifluoromethoxy)-poly(perfluoromethoxy)]-poly(perfluoroethanol)-difluoroacetic acid (Table 1). Fluorine incorporations of ˜20-27 (w/w)% were routinely attained for these substrates. Yields of emulsan were determined after ether extraction and hot phenol extraction and ranged from 61 to 135 mg per 100 mL of medium. This compares with growth of the bacterium on ethanol (1%) where 103 mg of polymer is produced per 100 mL (Table 2). In many cases the level of emulsan production was comparable on the fluorinated substrates and in the case of incorporation with retention of fluorine, in one case the yield of polymer was higher although not statistically significant.

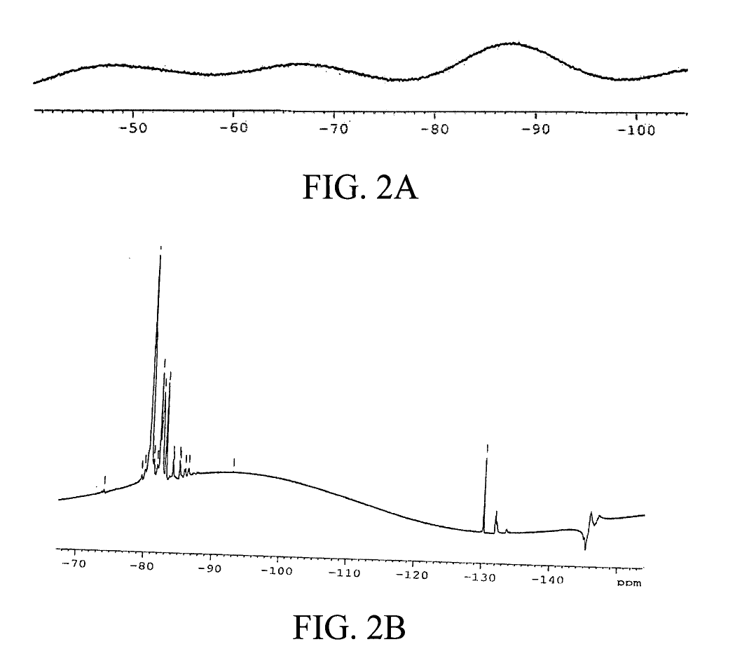

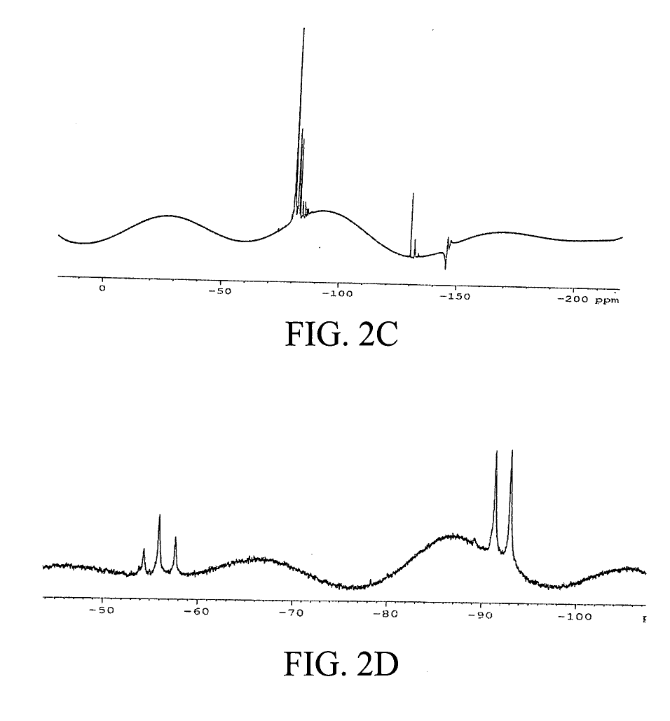

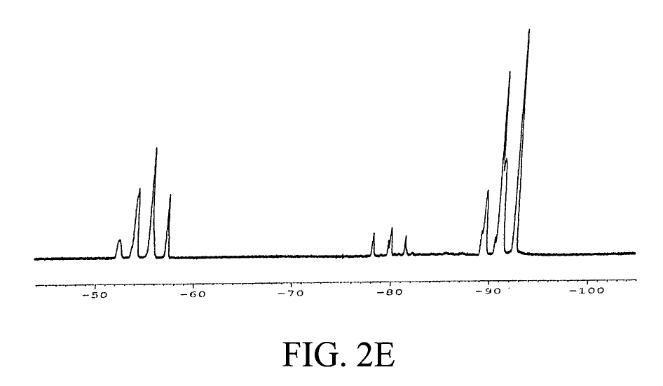

Since cell growth on the fluorinated compounds could be due to metabolism of the compounds along with the co-supplied ethanol, the retention of the fluorine on the polymer was determined. Incorporation was assessed by ¹⁹F-NMR of the purified polymers and by GC-MS of the hydrolyzed fatty acids. ¹⁹F-NMR spectra were obtained for the emulsans generated with the 2-poly(perfluoropropanoxy)-methylperfluoro-propanoate plus ethanol and the 2-[(carboxydifluoromethoxy)-poly(perfluoromethoxy)]-poly(perfluoro-ethanol)-difluoroacetic acid plus ethanol, along with the fluorinated compounds (monomers) (FIG. 2). The chemical shift for the incorporated 2-poly(perfluoropropanoxy)-methylperfluoropropanoate in emulsan was observed at −82.4 ppm, corresponding to the pure standard sample (FIG. 2b & c). In the case of 2-[(carboxydifluoromethoxy)-poly(perfluoromethoxy)]-poly(perfluoroethanol)-difluoroacetic acid the chemical shift due to incorporation was observed at −92.5 ppm (FIG. 2d & e).

Fatty acid profiles (by GC-MS) of the hydrolyzed fatty acids are shown in Table 4. The fatty acid content was lower in all cases for the bacterium grown on the 2-poly(perfluoropropanoxy)-methyl perfluoro propanoate and 2-[(carboxydifluoromethoxy)-poly(perfluoromethoxy)]-poly(perfluoroethanol)-difluoro-acetic acid than those in the control. Total fatty acid content per mg of emulsan was 285 and 165 nmol when grown on minimal medium with ethanol and 2-poly(perfluoropropanoxy)-methylperfluoropropanoate, and 2-[(carboxy-difluoromethoxy)-poly(perfluoromethoxy)]-poly(perfluoroethanol)-difluoroacetic acid, respectively. These levels contrast with emulsan generated on defined media with 1% ethanol only, where 443 nmol of fatty acids was present per mg of emulsan. The fluorofatty acid detected in 2-poly(perfluoropropanoxy)methylperfluoropropanoate-grown cells corresponded most closely to C16:0 F and represented 8.5 mol % of the total fatty acids present on the polysaccharide backbone. In the case of 2-[(carboxy-difluoromethoxy)-poly(perfluoromethoxy)]-poly(perfluoroethanol)-difluoroacetic acid-grown cells the sole fluorofatty acid detected most closely corresponded to 18:0 F and represented 5 mol % of the total fatty acids (Table 4).

An emulsification assay provided a semi-quantitative estimate of solution behavior. The emulsan sample generated by growth of the bacterium on minimal medium containing ethanol and 2-poly(perfluoro-propanoxy)methylperfluoropropanoate produced 300 Klett units (assayed on dodecane), compared to 190 and 50 units for emulsans when produced on minimal medium containing ethanol and 2-[(carboxy-difluoromethoxy)-poly(perfluoromethoxy)]poly(perfluoroethanol)-difluoroacetic acid and on ethanol only, respectively (Table 3).

It was previously known that the biological synthesis of emulsans enriched in specific chain lengths of fatty acids, hydroxylated fatty acids, or the degree of substitution by fatty acids along the polysaccharide backbone can be achieved by A. calcoaceticus RAG-1 (Zhang et al 1999). However, it was unexpected that fluorinated substrates can be incorporated. This invention has shown the successful incorporation of fluorinated compounds into emulsan, suggesting a new route to generating a broader family of amphilphilic polymers related to emulsans. This finding is quite significant in that the results suggest that this incorporation leads to substantive changes in solution behavior as determined by emulsification properties.

The biological synthesis of complex polymers containing fluorinated side chains along a hydrophilic backbone provides a novel strategy upon which to design and synthesize a series of polymers that contain components (e.g., main chain, pendant chains) that would otherwise be incompatible. While procedures for the chemical synthesis of fluoropolymers are available, these tend to be time consuming and complex.

TABLE 1
Chemical structures of the fluorinated compounds.

| Compound | Chemical structure | M_{w} |
|---|---|---|
| Poly(perfluoroethylene | HO₂CCF₂O[CF₂CF₂O]ₙCF₂OCF₂CO₂H | ˜600 |
| glycol)- | | |
| difluoromethyleneoxide-α,ω- | | |
| bis(carboxylic acid) | | |
| 2-Poly(perfluoropropanoxy)- | F[CF(CF₃)CF₂O]ₙCF(CF₃)CO₂CH₃ | ˜1,000 |
| methyl perfluoropropanoate | | |
| Poly(perfluoroethylene) | F[CF₂CF₂]CH₂CH₂OOC(C₁₇CH₃₅) | ˜700 |
| propyl-stearate | | |
| Poly(perfluoroethylene)-α,ω- | HOCH₂CF₂O[CF₂CF₂]ₙCF₂OCF₂CH₂OH | ˜500 |
| bis-(2-difluoroethanol) | | |
| Polytetrafluoroethyleneoxide- | CH₃O₂CF₂O(CF₂CF₂O)ₓ(CF₂O)_{y}CF₂CO₂CH₃ | ˜2,000 |
| co-difluoromethyleneoxide- | | |
| α,ω-bis(methylcarboxylate) | | |
| Poly(2-difluoroethylethoxy)- | HO(CH₂CF₂O)ₓ(CF₂CF₂O)_{y}(CF₂O)_{z}CF₂CH₂OH | ˜1,000 |
| poly-(perfluoroethyl)- | | |
| poly(perfluoro-methyleneoxy)- | | |
| 2-difluoroethanol | | |
| Poly(ethylethoxy)-poly-(2- | HO(CH₂CH₂O)ₓ(CH₂CF₂O)_{y}(CF₂CF₂O)_{z}CF₂CH₂OCH₂CH₂OH | ˜1,000 |
| difluoro-ethylethoxy)- | | |
| poly(perfluoroethyl-ethoxy)-2- | | |
| fluoroethoxyethanol | | |
| 1-3 Propyldiol-poly(ethyl-2- | HOCH₂CH₂CH₂O(CH₂CF₂O)_{y}(CF₂CF₂O)_{z}CF₂CH₂OCH₂CH₂CH₂OH | ˜300 |
| difluoroethoxy)- | | |
| poly(perfluoro-ethanol)-2- | | |
| difluoroethyl-3-propyldiol | | |
| 2-[2-Poly(carboxydifluoro- | HO₂CCF₂O[CF₂CF₂O]ₓ(CF₂O)_{Y}CF₂COOH | ˜2,000 |
| methoxy)- | | |
| poly(perfluoromethoxy)]- | | |
| poly(per-fluoroethanol)-α,ω- | | |
| bis(1,1-difluoro-acetic acid) | | |
| Methylperfluorohexadeconate | CF₃(CF₂)₁₄CO₂CH₃ | 887 |
| Perfluoro-3,6,9- | CF₃(CF₂)₃O(CF₂CF₂O)₂CF₂CO₂CH₃ | 576 |
| trioxatridecanoic acid, methyl | | |
| ester | | |
| Perfluoro-3,5,5′-trimethyl- | (CF₃)₃CCF₂CF(CF₃)CF₂COOH | 464 |
| hexanoic acid | | |

TABLE 2
Formation of emulsans on fluorinated substrates by A. calcoaceticus RAG-1.

| | | | | After hot | |
|---|---|---|---|---|---|
| | | | After ether | phenol | |
| | | | extraction³ | treatment⁴ | Fluorine- |
| Example | Compound¹ | Growth² | (mg) | (mg) | Incorporation⁵ |
| 1 | Ethanol + Poly | No growth | 0 | 0 | No |
| | (perfluoroethylene glycol) | | | | |
| | difluoromethyleneoxide-α,ω- | | | | |
| | bis(carboxylic acid) | | | | |
| 2 | Ethanol + Poly | No growth | 0 | 0 | No |
| | (perfluoroethylene) | | | | |
| | propylstearate | | | | |
| 3 | Ethanol + Poly | + | 68 ± 10 | 25 ± 5 | No |
| | (perfluoroethylene)-α,ω- | | | | |
| | bis-(2-difluoroethanol) | | | | |
| 4 | Ethanol + Poly | + | 100 ± 14 | 64 ± 7.5 | No |
| | (perfluoroethylene)-α,ω- | | | | |
| | bis-(2-difluoroethanol) | | | | |
| 5 | Ethanol + 2-Poly(perfluoro- | + | 104 ± 13 | 62 ± 6 | Yes |
| | propanoxy)methylperfluoro- | | | | |
| | propanoate | | | | |
| 6 | Only Ethanol | + | 103 ± 27 | 73 ± 31 | No |
| 7 | Ethanol + Poly | + | 65 ± 6 | 19 ± 6.5 | No |
| | (perfluoroethylene)-α,ω- | | | | |
| | bis(2-difluoroethanol) | | | | |
| 8 | Ethanol + poly(2- | + | 91 ± 14 | 35 ± 5 | No |
| | difluoroethyl-ethoxy)- | | | | |
| | poly(perfluoroethyl)-poly- | | | | |
| | (perfluoromethyleneoxy)-2- | | | | |
| | difluoroethanol | | | | |
| 9 | Ethanol + poly(ethylethoxy)- | + | 61 ± 9 | 17 ± 4 | No |
| | poly-(2-difluoroethylethoxy)- | | | | |
| | poly(perfluoro-ethylethoxy)-2- | | | | |
| | fluoroethoxyethanol | | | | |
| 10 | 1,3-Propyldiol-poly(ethyl-2- | + | 91 ± 20 | 31 ± 4.5 | No |
| | difluoroethoxy)- | | | | |
| | poly(perfluoro-ethanol)-2- | | | | |
| | difluoroethyl-3-propyl-diol | | | | |
| 11 | Ethanol + Polytetrafluoroethyleneoxide- | + | 135 ± 11 | 45 ± 5 | Yes |
| | co-difluoromethyleneoxide- | | | | |
| | α,ω-bis(methylcarboxylate) | | | | |

¹Average of three replicates, all media contained 1% ethanol plus 1% (w/v) organofluoro compound in minimal media (except ethanol control with organofluoro compound).
²(+) = growth observed; (−) = no growth observed.
³Emulsan produced after 6 days in culture and after ether extraction to remove non-covalently bound fatty acids.
⁴Emulsan after hot phenol extraction to remove proteins.
⁵Fluorine incorporation determined by ¹⁹F-NMR.

TABLE 3
Emulsification behavior of 3 different emulsans assayed on three
different alkanes (hexadecane, tetradecane and dodecane)
(average and standard deviation).

| | Emulsification Activity (Klett Units) | | |
|---|---|---|---|
| Carbon Source(s) | substrate | | |
| Used to Grow Polymer | n-hexadecane | n-tetradecane | n-dodecane |
| Ethanol | 125 ± 25 | 75 ± 5 | 50 ± 13 |
| 1% Ethanol + 2- | 120 ± 5 | 240 ± 10 | 300 ± 15 |
| Poly(perfluoro- | | | |
| propanoxy)methylperfluoro- | | | |
| propanoate (Example 5) | | | |
| 1% Ethanol + Polytetrafluoroethyleneoxide- | 90 ± 5 | 90 ± 10 | 190 ± 12 |
| co-difluoromethyleneoxide- | | | |
| α,ω-bis(methylcarboxylate | | | |
| (Example 11) | | | |

TABLE 4
Fatty acid composition of the fluorinated
emulsans produced by A. calcoaceticus
RAG-1 based on GC-MS analyses.

| | Fatty acid composition | |
|---|---|---|
| | (mol % [nmol/mg]) | |
| | Example 5 | Example 11 |
| Emulsan | 2-Poly(perfluoro- | Polytetrafluoroethyleneoxide- |
| Fatty acid | propanoxy)methyl | co-difluoromethyleneoxide- |
| substituents | perfluoro propanoate | α,ω-bis(methylcarboxylate |
| C12:0 | 6(18) | 35(59) |
| C12:0,2-OH | 13(38) | 16(26) |
| C12:0,3-OH | 0 | 0 |
| C14:0 | 2(5) | 23(38) |
| C14 OH | 3(9) | 0 |
| C16:0 | 4(12) | 9(15) |
| C16:0 F* | 8.5(24) | 0 |
| C17:0 | 7.5(21) | 0 |
| C18:0 | 39(110) | 0 |
| C18:0 F* | 0 | 5(8) |
| C19:0 | 17(48) | 12(19) |
| Unidentified fatty | 0 | 0 |
| acids* | | |
| Total fatty acids | 100(285) | 100(165) |

*Fluorofatty acid

Use of new Fluoroemulsans

Polymeric emulsifiers generally provide useful solution properties to enhance oil-in-water stability, emulsification specificity and have potential applications in food, paper, paint, bioremediation, agriculture, detergent and cosmetic industries. Fluoropolymers, in particular, display interesting physical and chemical properties, e.g., resistance to chemicals and unique surface properties due to low surface energy. Fluoropolymers can be used to achieve: low surface energy, chemical and moisture resistance, oil/grease resistance, adhesion to low-energy surfaces, low refractive index, surface lubricity, soil and dirt resistance.

The new compositions of this disclosure display novel surfactant and emulsification properties. Fluoroemulsans have many applications, including: (i) increasing the surface area of hydrophobic water-insoluble growth substrates; (ii) increasing the bioavailability of hydrophobic substrates by increasing their apparent solubility or desorbing them from surfaces; (iii) stabilizing membrane proteins in aqueous solution; (iv) drug delivery; (v) antimicrobial properties and (vi) regulating the attachment and detachment of microorganisms to and from surfaces. Of these, the use as antimicrobial agents is of particular importance (Viscardi G, Quagliotto P, Barolo C, Savarino P, Bami E, Fisicaro E., J. Org. Chem., 65(24), 8197-203, 2000.). The solubilization and emulsification of toxic chemicals by biosurfactants have also been reported (Banat IM, Makkar RS, Cameotra SS., Appl. Microbiol. Biotechnol., 53(5), 495-508, 2000). The biodegradability of fluoroemulsans provides several important advantages over chemical surfactants and opens many potential commercial applications such as bioremediation of oil-polluted soil and water, enhanced oil recovery, replacement of chlorinated solvents used in cleaning-up oil-contaminated pipes, vessels and machinery, use in the detergent industry, formulations of herbicides and pesticides and formation of stable oil-in-water emulsions for the food and cosmetic industries.

Fluoropolymers are uniquely characterized by very strong intramolecular bonds and very weak intermolecular interactions. This results in a combination of exceptional thermal, chemical and biological inertness, low surface tension, high fluidity, excellent spreading characteristics, low solubility in water, and high gas dissolving capacities, which are the basis for innovative applications in the biomedical field. Perfluoroalkyl chains are larger and more rigid than their hydrogenated counterparts. They are considerably more hydrophobic, and are lipophobic as well. Fluorinated surfactants are significantly more surface active than their hydrocarbon counterparts. Despite this, they are less hemolytic and less of a detergent. Fluorosurfactants appear unable to extract membrane proteins. Fluorinated chains confer to surfactants a powerful driving force for collecting and organizing at interfaces. As compared to non-fluorinated analogs, fluorosurfactants also have a much stronger capacity to self-aggregate into discrete molecular assemblies when dispersed in water and other solvents. Even very short, single-chain fluorinated amphiphiles can form highly stable, heat-sterilizable vesicles, without the need for supplementary associative interactions. Sturdy microtubules were obtained from non-chiral, non-hydrogen bonding single-chain fluorosurfactants. Fluorinated amphiphiles can be used to engineer a variety of colloidal systems and manipulate their morphology, structure and properties. Stable fluorinated films, membranes and vesicles can also be prepared from combinations of standard surfactants with fluorocarbon/hydrocarbon diblock molecules. In bilayer membranes made from fluorinated amphiphiles the fluorinated tails segregate to form an internal Teflon-like hydrophobic and lipophobic film that increases the stability of the membrane and reduces its permeability. This fluorinated film can also influence the behavior of fluorinated vesicles in a biological milieu. For example, it can affect the in vivo recognition and fate of particles, or the enzymatic hydrolysis of phospholipid components. Major applications of fluorocarbons currently in advanced clinical trials include injectable emulsions for delivering oxygen to tissues at risk of hypoxia; a neat fluorocarbon for treatment of acute respiratory failure by liquid ventilation; and gaseous fluorocarbon-stabilized microbubbles for use as contrast agents for ultrasound imaging. Fluorosurfactants also allow the preparation of a range of stable direct and reverse emulsions, microemulsions, multiple emulsions, and gels, some of which may include fluorocarbon and hydrocarbon and aqueous phases simultaneously. Highly fluorinated systems have potential for the delivery of drugs, prodrugs, vaccines, genes, markers, contrast agents and other materials.

The present fluorinated emulsans can be also be useful for drug delivery, diagnostics, as imaging or contrast agents, and as emulsifiers for blood substitutes. They thus include, but are not limited to pharmaceutical drugs, immunoadjuvants, vaccines, and the like. The fluorine-modified biopolymers of the instant invention are useful as diagnostic tools. The methods of the instant invention permit the preparation of diagnostic agents with dual functionalities. Thus, the simultaneous incorporation of ¹⁹F or superparamagnetic residues and fluorescent moieties into biopolymers affords diagnostic probes that can be employed for both MRI and fluorescent studies. Examples of such dual function diagnostic probes are those biopolymers that contain both a fluorine moiety as described herein and a fluorescent moiety or a fluorinated fluorescent moiety such as: 4-trifluoromethyl-7-aminocoumarin, 4-trifluoromethyl-umbelliferone (or its acetate or butyrate derivatives), 4-fluoro-7-sulfamyl-benzofurazam, certain BODIPY dyes, e.g., N-(4,4′-difluoro-5,7-dimethyl-4-bora-3a,4a-diaza-s-indacene-3-yl)-methyliodoacetamide, N-(4,4′-difluoro-1,3,5,7-tetramethyl-4-bora-3a,4a-diaza-s-indacene-2-yl)-iodoacetamide and 4,4′-difluoro-5-phenyl-4-bora-3a,4a-diaza-s-indacene-3-propionic acid, 3-chloro-1-(3-chloro-5-(trifluoromethyl)-2-pyridimyl)-5-(trifluoro-methyl)-2[1H]-pyridinone, 6-carboxymethylthio-2′,4,′5,7′-tetrabromo-4,5,7-trifluorofluorescein (Eosin F3S), and Oregon Green carboxylic acid.

In additional embodiments halogenated emulsans as described herein that contain a halogen moiety other than fluorine or in combination with a fluorine moiety provide useful solution properties to enhance oil-in-water stability, emulsification specificity and have potential applications in food, paper, paint, bioremediation, agriculture, detergent and cosmetic industries.

I claim:

1. A method of making emulsans which comprises culturing a microorganism capable of producing emulsans in an effective emulsan producing culture medium wherein the culture medium contains fluorinated compounds that are assimilated by the microorganism and incorporated into the emulsan molecule elaborated by the microorganism.

2. The method of claim 1 wherein the microorganism is a bacteria.

3. The method of claim 2 wherein the bacteria is an Acinetobacter.

4. The method of claim 2 wherein the bacteria is Acinetobacter calcoaceticusRAG1.

5. The method of claim 4 wherein the fluorinated compounds are fluorinated fatty acids.

6. The method of claim 5 wherein the fluorinated fatty acids are 2-poly(perfluoropropanoxy)-methylperfluoropropanoate; 2-[(carboxydifluoromethoxy)-poly(perfluoromethoxy)]-poly(perfluoroethanol)-difluoroacetic acid; or a mixture of both.

7. The method of claim 6 wherein the fluorinated fatty acid is 2-poly(perfluoropropanoxy)-methylperfluoropropanoate.

8. The method of claim 6 wherein the fluorinated fatty acid is 2-[(carboxydifluoromethoxy)-poly(perfluoromethoxy)]-poly(perfluoroethanol)-difluoroacetic acid.

9. A method of making fluorinated emulsans which comprises culturing an emulsan producing organism in the presence of fluorinated compounds that are assimilated by the microorganism and incorporated into the emulsan molecule elaborated by the organism.

10. The method of claim 9 wherein the emulsan producing organism is of the genus Acinetobacter.

11. The method of claim 9 wherein the organism is Acinetobacter calcoaceticus RAG1.

12. The method of claim 11 wherein the fluorinated compounds are fluorinated fatty acids are 2-poly(perfluoropropanoxy)-methylperfluoropropanoate; 2-[(carboxydifluoromethoxy)-poly(perfluoromethoxy)]-poly(perfluoroethanol)-difluoroacetic acid; or a mixture of both.

13. In a method of making emulsans by culturing an emulsan producing microorganism in a culture medium under conditions sufficient to produce emulsans, the improvement which comprises:
adding one or more fluorinated compounds to the culture medium whereby the fluorinated compounds are assimilated by the microorganism and incorporated into the emulsan molecule resulting in fluorinated emulsans.

14. The improved method of claim 13 wherein the fluorinated compound is 2-[(carboxydifluoromethoxy)-poly(perfluoromethoxy)]-poly(perfluoroethanol)-difluoroacetic acid.

15. The improved method of claim 13 wherein the fluorinated compound is 2-poly(perfluoropropanoxy)-methylperfluoropropanoate.

16. A fluorinated emulsan polymer made by a process which comprises fermenting a microorganism capable of producing emulsans in an effective emulsan producing culture medium wherein the culture medium contains fluorinated compounds that are assimilated by the microorganism and incorporated into the emulsan molecule elaborated by the microorganism.

17. A polysaccharide backbone fluorinated emulsan polymer made by a process which comprises fermenting Actinobacter calcoaceticus RAG1 in an effective emulsan producing culture medium wherein the culture medium contains one or more fluorinated carboxylic acid compounds or esters thereof that are assimilated by the microorganism and incorporated into the emulsan molecule made by the microorganism whereby the fluorinated carboxylic acid or fluorinated carboxylic acid ester is attached to the polysaccharide backbone of the emulsan.

18. The polysaccharide of claim 17 wherein the fluorinated carboxylic acid is 2-[(carboxydifluoromethoxy)-poly(perfluoromethoxy)]-poly(perfluoroethanol)-difluoroacetic acid.

19. The polysaccharide of claim 17 wherein the fluorinated carboxylic acid is 2-poly(perfluoropropanoxy)-methylperfluoropropanoate.

20. An isolated fluorinated emulsan containing a polysaccharide backbone produced by the fermentation of Actinobacter calcoaceticus RAG1 in the presence of one or more fluorinated carboxylic acid compounds or fluorinated carboxylic acid ester compounds whereby said fluorinated compounds are attached to the polysaccharide backbone of the emulsan.

21. The emulsan of claim 20 wherein the fluorinated carboxylic acid is 2-[(carboxydifluoromethoxy)-poly(perfluoromethoxy)]-poly(perfluoroethanol)-difluoroacetic acid.

22. The emulsan of claim 20 wherein the fluorinated carboxylic acid is 2-poly(perfluoropropanoxy)-methylperfluoropropanoate.

23. A method of making emulsans which comprises culturing a microorganism capable of producing emulsans in an effective emulsan producing culture medium wherein the culture medium contains as the carbon source (a) a lower alcohol and (b) fluorinated compounds that are assimilated by the microorganism and incorporated into the emulsan molecule elaborated by the microorganism.

24. The method of claim 23 wherein the fluorinated carboxylic acid is 2-[(carboxydifluoromethoxy)-poly(perfluoromethoxy)]-poly(perfluoroethanol)-difluoroacetic acid.

25. The method of claim 23 wherein the fluorinated carboxylic acid is 2-poly(perfluoropropanoxy)-methylperfluoropropanoate.

26. In a method of making emulsans by culturing an emulsan producing microorganism in a culture medium containing a carbon source under conditions sufficient to produce emulsans, the improvement which comprises employing as the carbon source:
a. a lower alcohol and
b. one or more fluorinated compounds whereby the fluorinated compounds are assimilated by the microorganism and incorporated into the emulsan molecule resulting in fluorinated emulsans.

27. The improved method of claim 26 wherein the fluorinated compound is 2-[(carboxydifluoromethoxy)-poly(perfluoromethoxy)]-poly(perfluoroethanol)-difluoroacetic acid.

28. The improved method of claim 26 wherein the fluorinated compound is 2-poly(perfluoropropanoxy)-methylperfluoropropanoate.

29. An emulsan compound of the formula 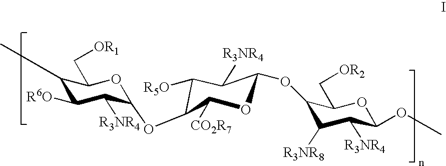
wherein
R₁═H, acyl, X; R₂═H, acyl, X; R₃═H, COCH₃, X; R₄═H, X COX, CH₂X, CH₂NHX; R₅═H, X; R₆═H, X; R₇═H, X, COCH₃, COX
X=haloalkyl, haloaryl, haloacyl, perhaloalkyl, perhaloaryl, perhaloacyl, perhalopolymer fluoroalkyl, fluoroaryl, fluoroacyl, perfluoroalkyl, perfluoroaryl, perfluoroacyl, perfluoropolymer, F, Cl, Br, I, CF₃, CCl₃ COCₓF_{y}, CH₂Br, CH₂I, CₓF_{y}H_{z}, COCH₂(CₓH_{3-y}Br_{y}), COCH₂(CₓH_{3-y}Cl_{y}), COCH₂(CₓH_{3-y}I_{y}), CₓF_{y}H_{z}, ([CH₂]ₘO)ₓ(CH₂CF₂O)_{y}(CF₂CF₂O)_{z}(CF₂)₂CF₂CH₂O(CH₂)ₚOH, CH₂C(OH)CₓF_{y}H_{z}, CₓF_{y}H_{z}Oₚ, COCₓF_{y}H_{z}, OCH₂CₓF_{z}[CₓF_{z}O]ₘF, CH₂C(CH₃)CO₂CₓH_{z}(CF₂)ₘCF₃, CH₂(CF₂O)ₓ(CF₂CF₂O)_{y}(CF₂O)_{z}CF₂CH₂OH, COCF(CF₃)-[CF(CF₃)CF₂O]ₘF, NHCₓF_{y}H_{z}Oₚ, CH₂CF₂O[CF₂CF₂O]ₘ(CF₂OCF₂CH₂OH, COCₓH_{z}(CF₂)ₘCF₃, COCF₂O[CF₂CF₂O]ₙCF₂OCF₂CO₂H, ([CH₂]ₘO)ₓ(CH₂CF₂₀)_{y}(CF₂CF₂O)_{z}CF₂CH₂O(CH₂)ₚOH, N[CₓF_{y}H_{z}]ₚ, CₓH_{z}CO₂CₓH_{z}(CF₂)ₘCF₃, COCₓF_{y}[CₚF_{z}O]ₘF, a luminescent residue, a fluorescent residue, a halogenated luminescent residue or a halogenated fluorescent residue and m, x, p, y and, z are integers from 1 to 150.

30. The emulsan compound of claim 29 wherein m is from 10 to 100 inclusive; x, p, y, z are from 10 to 75 inclusive; acyl and alkyl residues are lipophilic moieties, selected from the group consisting of saturated and unsaturated aliphatic residues with Cₖ chains wherein k is from 2 to 100 inclusive; and aryl residues are aromatic moieties selected from the group consisting of benzyl, biphenyl, phenyl polycyclic aromatics, and heteroatom-containing aromatics.

31. The emulsan compound of claim 30 wherein m is from 10 to 50 inclusive; x, p, y, z are from 2 to 50 inclusive.

32. The emulsan compound of claim 31 wherein x, p, y, z are from 10 to 50 inclusive and k is from 2 to 20 inclusive.
